(19) United States
(12) Patent Application Publication
(10) Pub. No.: US 2015/0182140 A1
(43) Pub. Date: Jul. 2, 2015

(54) ARTERIAL PULSE ANALYSIS METHOD AND SYSTEM THEREOF

(71) Applicant: Industrial Technology Research Institute, Chutung (TW)

(72) Inventors: Chuan-Wei Ting, Chutung (TW); Ming-Yen Chen, Chutung (TW)

(73) Assignee: INDUSTRIAL TECHNOLOGY RESEARCH INSTITUTE, Chutung (TW)

(21) Appl. No.: 14/327,808

(22) Filed: Jul. 10, 2014

(30) Foreign Application Priority Data
Dec. 30, 2013 (TW) 102148975

(51) Int. Cl. A61B 5/024

(52) U.S. Cl. CPC A61B 5/02444

(57) ABSTRACT

An arterial pulse analysis method and a related system are provided. The arterial pulse analysis method segments a continuous pulse signal into a plurality of single pulses, processes at least one of the single pulses to obtain non-time series data corresponding to the at least one of the single pulses, and processes the non-time series data of the at least one of the single pulses with a multi-modeling algorithm to obtain at least one feature point of the at least one of the single pulses.

### CROSS-REFERENCE TO RELATED APPLICATIONS

This application claims foreign priority under 35 U.S.C. §119(a) to patent application Ser. No. 10/214,8975, filed on Dec. 30, 2013, in the Intellectual Property Office of Ministry of Economic Affairs, Republic of China (Taiwan, R.O.C.), the entire content of which patent application is incorporated herein by reference.

### TECHNICAL FIELD

The present disclosure relates to an arterial pulse analysis method and system thereof, and, more particularly, to an arterial pulse analysis method and system that are capable of analyzing the status of a cardiovascular system.

### BACKGROUND OF THE INVENTION

Cardiovascular disease is one of the major diseases of modern people, and thus how to effectively assess the state of a cardiovascular system has been one of the important subjects. Arterial pulse signals are a physiological parameter obtained mainly by measuring variations in the blood and the arteries of a measured body part in the cardiac cycles. Although the arterial pulse signals are subjected to the influences of physiological factors, such as cardiac output, arterial wall elasticity, blood volume, vascular resistance of the peripheral arteries and the arterioles, blood viscosity and the like, they remain one of the popular technical means for assessing the state of the cardiovascular system due to the simplicity and ease of operation of the arterial pulse signals analysis and equipment.

Continuous arterial pulse signals can be obtained by non-intrusive measurement devices. With advances in measurement technology, even mobile devices with their built-in sensors, such as built-in camera lens and flash, are capable of obtaining arterial pulse signals, and further analyzing and assessing physiological health information, such as the heart rate and other cardiovascular parameters. However, the majority of today's non-invasive arterial pulse measurement equipment, such as pressure-type wrist sphygmomanometers, sphygmography, optical oximeters, are vulnerable to movement and gestures of the human subjects, surrounding light, temperature and other factors during measurement. These may interfere with the measured signal quality, leading to deviations in the measured continuous arterial pulse signals and forming non-standard forms of arterial pulse signals. Such non-standard forms of arterial pulse signals usually have no obvious dicrotic notch, or have multiple peaks.

Therefore, there is a need for a technical means to handle non-standard forms of arterial pulse signals.

### SUMMARY OF THE INVENTION

The present disclosure provides an arterial pulse analysis method, comprising:

obtaining a continuous pulse signal through an arterial pulse measuring device; segmenting the continuous pulse signal into a plurality of single pulses; performing a data pre-processing step on at least one of the single pulses to obtain non-time series data corresponding to the at least one of the single pulses; and processing the non-time series data of the at least one of the single pulses with a multi-modeling algorithm to obtain at least one feature point corresponding to the at least one of the single pulses.

The present disclosure provides an arterial pulse analysis system, comprising: a signal acquisition unit for generating a continuous pulse signal; and an operation unit, including: a pulse segmentation module for processing the continuous pulse signal to segment the continuous pulse signal into a plurality of single pulses; a pre-processing module for processing at least one of the single pulses to obtain non-time series data corresponding to the at least one of the single pulses; and a multi-modeling module for processing the non-time series data of the at least one of the single pulses to obtain at least one feature point corresponding to the at least one of the single pulses.

### BRIEF DESCRIPTION OF DRAWINGS

FIG. 1 is a flowchart illustrating an arterial pulse analysis method in accordance with one embodiment of the present disclosure.

FIG. 2 is a schematic diagram depicting feature points obtained after a multi-modeling algorithm is performed in accordance with one embodiment of the present disclosure.

FIGS. 3A, 3B and 3C are schematic diagrams depicting an arterial pulse analysis method in accordance with one embodiment of the present disclosure.

FIG. 4 is a flowchart illustrating an arterial pulse analysis method in accordance with another embodiment of the present disclosure.

FIGS. 5A, 5B and 5C are schematic diagrams depicting a data pre-processing step in accordance with one embodiment of the present disclosure processing a pulse.

FIG. 6 is a block diagram depicting an arterial analysis system in accordance with the present disclosure.

### DETAILED DESCRIPTION OF THE INVENTION

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

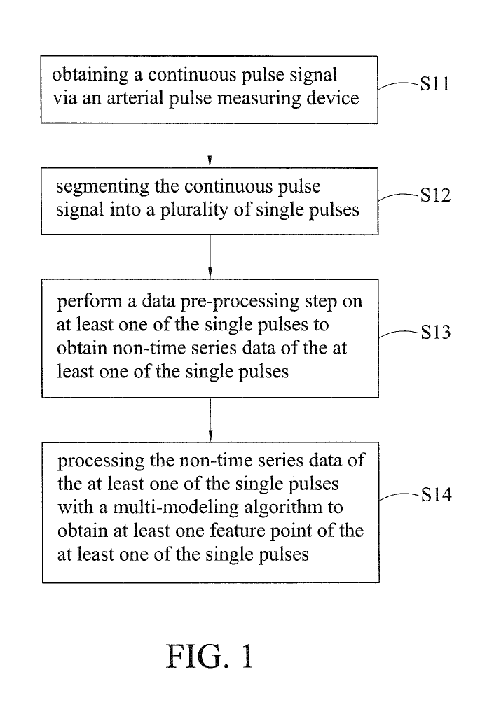

FIG. 1 is a flowchart illustrating an arterial pulse analysis method in accordance with one embodiment of the present disclosure. In step S11, a continuous arterial pulse signal is obtained through an arterial pulse measuring device. In an embodiment, the arterial pulse measuring device is, but not limited to, a sphygmomanometer, a sphygmography, an oximeter or a camera. The arterial pulse measuring device is pressure-type or optical-type, and is used for analyzing pressure changes or differences in light absorption of tissues of specific parts of the body to learn the changes of blood vessels and blood volume of the measured parts, and then converting the information into a continuous arterial pulse signal. An example of a pressure-type arterial pulse measuring device is a pressure-type wrist pulse pressure band with piezoelectric pressure sensors to capture pressure changes in the tested parts. An optical-type arterial pulse measuring device can irradiate a tested part with visible or infrared light, and then retrieve the changes in optical density of the tested part through a photodiode. More recently, a light sensing element (e.g., a CMOS or a CCD) in a camera is used as the light sensor instead of the aforesaid photodiode to detect the changes in optical density.

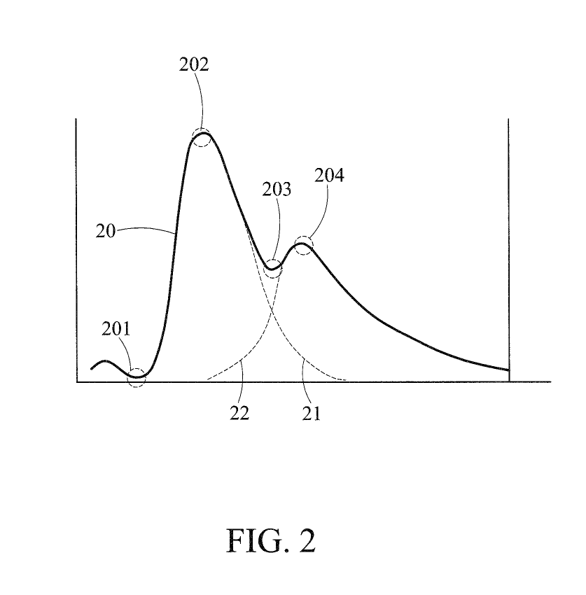

As shown in FIG. 2, the pulse 20 of the continuous pulse signal is, for example, an arterial pulse, also known as the blood pressure waveform, arterial blood pressure waveform, blood pressure pulse and the like. The term “arterial pulse” is used in the following description. The arterial pulse has several feature points that can be interpreted with meanings. For example, the pulse 20 has a plurality of feature points, such as pacemaker 201, percussion wave peak 202, dicrotic notch 203, and dicrotic wave peak 204. The pacemaker 201 represents the starting point of the waveform of an entire arterial pulse wave. The pacemaker 201 also refers to the blood pressure and volume at the end of the diastole of the heart or the starting point of ventricular ejection when the heart begins to contract and a large amount of blood begins to flow into the arteries. As a result, the intravascular volume and blood flow volume increase rapidly. At the end of the ventricular ejection period, the arterial pulse waveform rises dramatically until it reaches the percussion wave peak 202. This indicates the maximum vascular volume during systole when the blood vessel walls experience rapid expansion. The descending of the percussion wave peak 202 represents the gradual decreases in intravascular volume and blood flow volume, and the blood vessel walls are gradually retracted to the state before the expansion. The dicrotic wave peak 204 is a prominent peak when the percussion wave peak 202 is descending. It is a rebound wave as a result of brief fluctuations in blood volume in the arterial walls of a specific measured part of the body caused by the wave in the blood vessels being transmitted to the extremities and bounced back. A depression between dicrotic wave peak 204 and the percussion wave peak 202 is the dicrotic notch 203, which represents the arterial hydrostatic empty time, and is also a demarcation point for systole and diastole. These feature points can be used as physiological health indicators for assessing the heart rate and cardiovascular parameters. For example, the time intervals between the two wave peaks can be regarded as the RR interval (RRI) sequence of an electrocardiogram (ECG), and the physiological state of the user can be known by further heart rate variability (HRV) analysis. Moreover, through the arterial pulse patterns, the user's cardiac contractility, blood vessel elasticity, blood viscosity, vascular resistance of the peripheral arteries and the arterioles and other parameters that reflect the cardiovascular health of the user can be obtained.

The continuous pulse signal obtained in step S11 is composed by a number of single pulses. In order to analyze the feature points (e.g., the pacemaker, the percussion wave peak, the dicrotic notch, the dicrotic wave peak, etc.) of at least one single pulse, the continuous pulse signal is segmented into a plurality of single pulses (step S12). The segmentation method separates the single pulses by using peaks or valleys in the continuous pulse signal as segmenting points. Each single pulse represents the pulse generated by one beat of the heart.

After obtaining a plurality of single pulses, in step S13, a data pre-processing step is performed on at least one of the single pulses. After the data pre-processing step is performed, non-time series data corresponding to the at least one of the single pulses can be obtained. More specifically, the waveform of a normal pulse shows time series data with time-varying amplitudes. The horizontal axis usually represents the time, and the vertical axis represents the amplitude. The so-called non-time series data are obtained by segmenting (or grouping) the pulse waveform of the time series data into a plurality sets of data, unit time by unit time, wherein each data set corresponds to the value of the amplitude, and then converting the value of the amplitude originally represented by the vertical axis in each data set into frequency. As a result, a pulse waveform in the form of time series data having an amplitude-time representation is converted to non-time series data having set-frequency representation. Thus, the non-time series data is a series of data without time representation. In one implementation, the non-time series data can be plotted as a histogram, but the present disclosure is not limited thereto. In addition, the data pre-processing step is only required to be performed on at least one of the single pulses. The present disclosure does not require the data pre-processing step be performed on all of the single pulses at once, nor limits the number of single pulses processed each time. The data pre-processing step may also be performed on all of the single pulses at once.

Proceed to step S14, wherein a multi-modeling algorithm is used to process the non-time series data of the at least one of the single pulses in order to obtain at least one feature point corresponding to the at least one of single pulses. The so-called multi-modeling algorithm employs a Gaussian mixture model (GMM) to process the non-time series data of the at least one of the single pulses. A Gaussian mixture model is a combination of a plurality of Gaussian functions or Gaussian distributions according to different weights. In one embodiment of the present disclosure, a Gaussian mixture model includes at least two or more Gaussian functions, but the present disclosure is not limited thereto. In another embodiment of the present disclosure, the multi-modeling algorithm may also employ a plurality of triangular wave models to process the non-time series data of the at least one of the single pulses, or a mixture model of at least one Gaussian model and at least one triangular wave model to process the non-time series data of the at least one of the single pulses, but the present disclosure is not limited thereto. The characteristic values of the waveform (e.g., location of the wave peak) plotted by the Gaussian functions are the feature points of the pulse, such as the percussion wave peak and the dicrotic wave peak. In an embodiment, two Gaussian functions correspond to the percussion wave peak and the dicrotic wave peak, respectively. As shown in FIG. 2, the pulse 20 is represented by a first Gaussian function 21 and a second Gaussian function 22. The averages (i.e., the locations of the wave peaks) of the first Gaussian function 21 and the second Gaussian function 22 represent the vertices of the percussion wave peak and the dicrotic wave peak, respectively, and can thus be used as the feature points of the percussion wave peak 202 and the dicrotic wave peak 204 of the pulse 20. In addition, the characteristics values of the triangular wave model (e.g., the location of the wave peak) can also be used as the feature points of the pulse. Moreover, if a mixture algorithm involving both the Gaussian model and the triangular wave model is employed, then the feature points are the respective characteristics values of the Gaussian model (e.g., the location of the wave peak), characteristics values of the triangular wave model (e.g., the location of the wave peak), intersections of both waveforms of the Gaussian model and the triangular wave model in the mixture model, characteristics values of the Gaussian model in the mixture model, or the characteristics values of the triangular wave model in the mixture model. The above characteristic values of a Gaussian function can be statistics such as mean, standard deviation, median, mode, minimum, maximum, variability, skewness, kurtosis and/or the like that correspond to the feature points of the pulse. In addition, the characteristic values of a triangular wave model can be statistics such as vertex, height, width and/or the like that correspond to the feature points of the pulse. The intersections of both waveforms of the Gaussian model and the triangular wave model in the mixture model can be the intersections of any one of the characteristics values of the Gaussian function and any one of the characteristics values of the triangular wave model, or the characteristics values of the Gaussian model or the triangular wave model in the mixture model, but the present disclosure is not limited thereto. Furthermore, the step of using multi-modeling algorithm is only required on at least one of the single pulses; the present disclosure does not require the step of using multi-modeling algorithm to be done on all of the single pulses at once, nor limit the number of single pulses processed each time. The step of using multi-modeling algorithm may also be done on all of the single pulses at once.

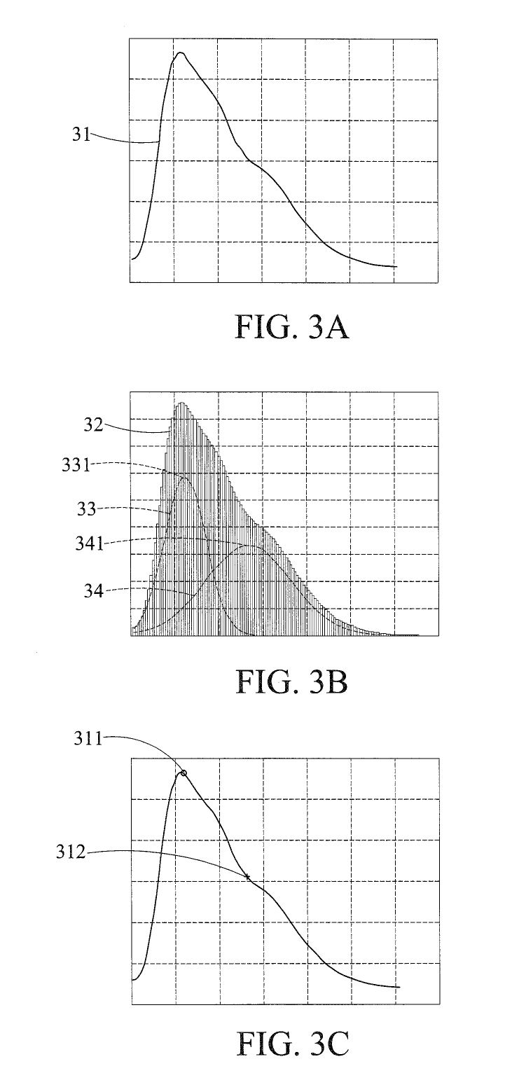

Refer to FIGS. 3A, 3B and 3C. FIG. 3A is a schematic diagram depicting a single pulse 31. As shown in FIG. 3B, the single pulse 31 is data pre-processed to form a pulse of non-time series 32. After multi-modeling algorithm processing on this pulse of non-time series 32, a first Gaussian function 33 and a second Gaussian function 34 are shown to represent the pulse of non-time series 32 (i.e., equivalent to the single pulse 31 in FIG. 3A), and the first Gaussian function 33 has a first vertex 331, and the second Gaussian function 34 has a second vertex 341. The first vertex 331 and the second vertex 341 corresponds to the pulse of non-time series 32 (i.e., equivalent to the single pulse 31 in FIG. 3A). The values on the horizontal axis of the pulse of non-time series 32 corresponding to the locations of the first vertex 331 and the second vertex 341 on the vertical axis are found. With the values on the vertical axis corresponding to the single pulse 31, two feature points—a percussion wave peak 311 and a dicrotic wave peak 312 of the single pulse 31—can be found (as shown in FIG. 3C). By processing the pulse of non-time series data with a multi-modeling algorithm, the locations of the feature points of the pulse can be effectively retrieved, and physiological state can be analyzed based on the meanings of the locations of these feature points, such as assessing cardiovascular health.

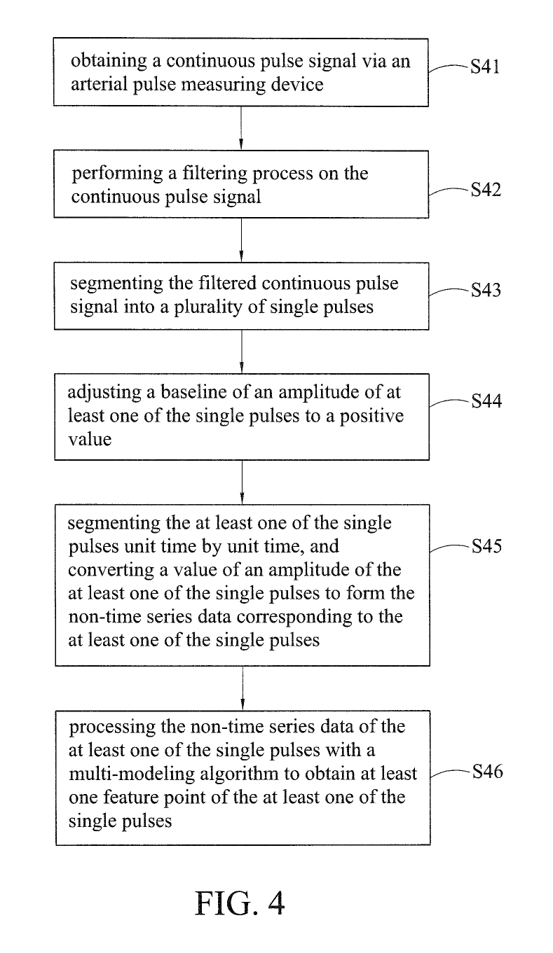

In another embodiment of the present disclosure, FIG. 4 shows a flowchart illustrating the arterial pulse analysis method in accordance with another embodiment of the present disclosure. Some steps described in this embodiment are the same as those described in the previous embodiment, and thus will not be repeated. In step S41, a continuous arterial pulse signal is obtained through an arterial pulse measuring device. Before the continuous arterial pulse signal is processed, a filtering process is performed (step S42). The filtering process is performed to eliminate the influence of non-cardiovascular factors in the continuous pulse signal. In an embodiment, the filtering process is a high-pass filter that eliminates low frequency noise, a low-pass filter that eliminates high frequency noise, or a bandpass filter that eliminates particular frequency bands.

In step S43, the filtered continuous pulse signal is segmented into a plurality of single pulses. The segmentation method may include separating the continuous pulse signal into a plurality of pulses by using peaks or valleys in the continuous pulse signal as segmenting points. After a plurality of single pulses are obtained, and before at least one of the single pulses is processed by a multi-modeling algorithm, the single pulses containing time data are first converted into non-time series data form suitable for multi-modeling, by performing the data pre-processing step. The data pre-processing step includes steps S44 and S45.

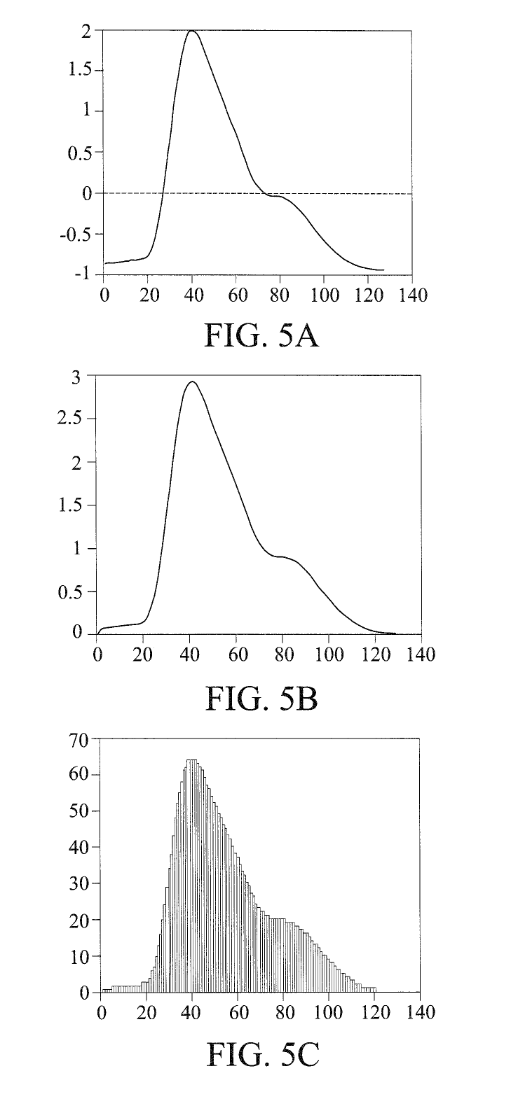

Refer to FIGS. 5A, 5B and 5C. FIG. 5A shows a waveform of the original pulse. The horizontal axis indicates the time, and the vertical axis indicates the amplitude. In step S44, the baseline of the amplitude of at least one of the single pulses is adjusted to positive values. That is, the waveform of the entire pulse shown in FIG. 5A is shifted upwards, such that the minimum of the amplitude of the pulse is not less than zero, as shown in FIG. 5B, The dashed line shown in FIG. 5A is shifted downwards to form the graph shown in FIG. 5B. Proceed to step S45. As shown in FIG. 5C, the pulse is segmented into a plurality sets of data unit time by unit time, with each time point (each set) corresponding to a value of amplitude. Then, the amplitude value corresponding to each time point is converted to frequency representation. For example, the vertical axis shown in FIG. 5C represents frequency. The conversion method may be carried out by amplifying the values of the amplitude, for example, the vertical-axis data of FIG. 5C are obtained by amplifying the vertical-axis data of FIG. 5B. However, the conversion method may also be carried out by reducing the amplitude values, or no conversion is carried out on the amplitude values. More specifically, conversion of the amplitude value corresponding to each time point into frequency can be carried out based on the amplitude characteristics of the single pulse. The amplitude characteristic refers to how much the amplitude of the pulse fluctuates. If the amplitude characteristic of a single pulse is not significant, it means that the amplitude of the pulse does not fluctuate dramatically, so conversion can be done by amplifying the amplitude values to facilitate subsequent analysis. If the amplitude characteristic of a single pulse is significant, it means that the amplitude of the pulse fluctuate dramatically, so conversion can be done by reducing the amplitude values or no conversion is done to facilitate subsequent analysis; the present disclosure is not limited as such.

After the data pre-processing step is performed, the at least one of the single pulse can be represented in set-frequency form instead of time-amplitude form, and the data can be plotted as non-time series data, such as in a histogram data distribution form, but the present disclosure is not limited thereto. As such, in step S46, a multi-modeling algorithm is used to process the non-time series data of the at least one of the single pulses in order to obtain at least one feature point corresponding to the at least one of single pulses, and the feature point can be used for physiological assessments, wherein the feature points is at least one of the pacemaker, percussion wave peak, dicrotic notch and dicrotic wave peak. If the multi-modeling algorithm employs a mixture model of at least one Gaussian model and at least one triangular wave model, for example, the intersection of the Gaussian model and the triangular wave model in the mixture model is used as the dicrotic notch. The characteristic of the Gaussian model in the mixture model is used as the percussion wave peak or the dicrotic wave peak. The characteristic of the triangular wave model in the mixture model is used as the percussion wave peak or the dicrotic wave peak. Therefore, if a mixture model is used, any one or a combination of any two types of feature points can be obtained, but the present disclosure is not limited as such.

Regardless it is the multi-modeling algorithm in step S14 or step S46, since the multi-modeling algorithm is a probabilistic multi-model, superimposed multi-model functions will satisfy “Axioms of Probability.” Satisfying probability axioms means satisfying its three axioms: (1) the probability of any event in the sample space is a positive real number or zero; (2) probability for each sample space is 1; and (3) if event A and event B in the sample space are mutually exclusive, then the probability of event A or event B occurring is the sum of their respective probabilities of event A and event B. In order to identify the Gaussian function that approximates the pulse the most, the Gaussian model is converged through Maximum Likelihood estimation and Expectation Maximization. The convergence thus requires less time, and increases the efficiency of retrieving the feature points of the arterial pulse. However, Maximum Likelihood estimation and Expectation Maximization can also be used on the triangular wave model, and also used for the convergence of the individual functions of the Gaussian model and the triangular wave model in the mixture model, and the present disclosure is not limited thereto.

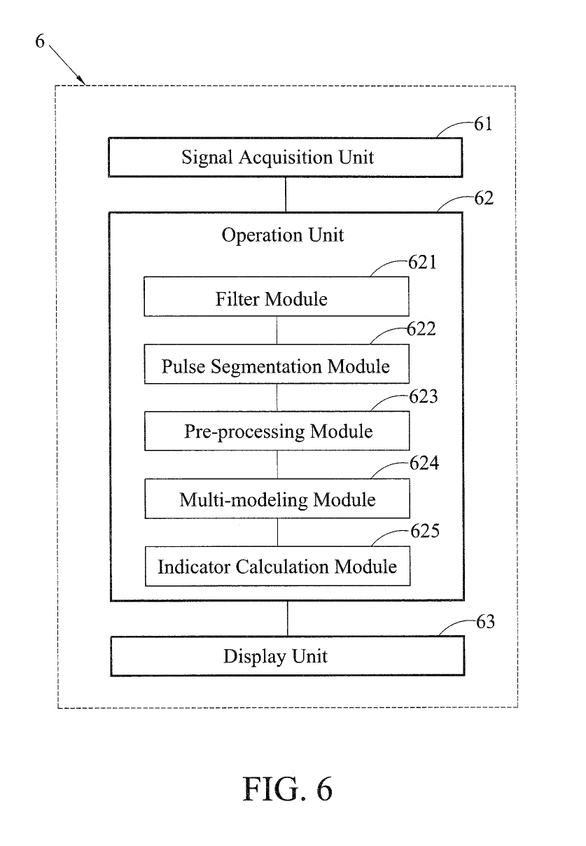

The present disclosure further provides an arterial pulse analysis system. Referring FIG. 6, an arterial pulse analysis system 6 includes a signal acquisition unit 61, an operation unit 62, and a display unit 63. The operation unit 62 includes a filter module 621, a pulse segmentation module 622, a pre-processing module 623, a multi-modeling module 624, and an indicator calculation module 625. It should be noted that these modules can include software, hardware, or a combination of the foregoing. Software can be, for example, mechanical codes, firmware, embedded codes, application software or a combination of the foregoing. Hardware can be, for example, circuits, processors, computers, integrated circuits, integrated circuit core, or a combination of the foregoing. The signal acquisition unit 61 is used for generating a continuous pulse signal. In an embodiment, the signal acquisition unit 61 can be a sphygmomanometer, a sphygmography, an oximeter or a camera, but the present disclosure is not limited thereto. After the signal acquisition unit 61 captures a continuous pulse signal of a body under test (e.g., a human being), the continuous pulse signal is sent to the filter module 621 to filter out the noise and to generate a filtered continuous pulse signal. The filter module 621 performs a high-pass filtering step that eliminates low frequency noise, a low-pass filtering step that eliminates high frequency noise, or a bandpass filtering step that eliminates a certain frequency band, and the present disclosure is not limited as such. The filtered continuous pulse signal is then passed to the pulse segmentation module 622 for segmenting the filtered continuous pulse signal into a plurality of single pulses. The pulse segmentation module 622 segments the filtered continuous pulse signal into a plurality of single pulses based on the valley or the peak of each pulse. At least one of the segmented single pulses is passed to the pre-processing module 623 for adjusting the baseline of the at least one of the single pulses to a positive value, so as to allow the at least one of the single pulses to be segmented unit time by unit time, and the amplitude values of the at least one of the single pulses to be converted, for example, by amplifying or reducing the amplitude values. As such, each segmented time point corresponds to a frequency converted from an amplitude value. As a result, a single pulse represented in a time-amplitude manner can be represented in a set-frequency manner, thereby forming a non-time series data corresponding to the single pulse. In one implementation, the non-time series data can be in the form of histogram data distribution, but the present disclosure is not limited to this. The multi-modeling module 624 is used for processing the non-time series data of the at least one of the single pulses to obtain at least one feature point corresponding to the at least one of the single pulses. The processing method includes using a Gaussian mixture model of at least two Gaussian functions, a plurality of triangular wave models or a mixture model to process the non-time series data of the at least one of the single pulses. The functionalities and technical means of the various modules and units in the arterial pulse analysis system 6 are the same as those described with respect to the arterial pulse analysis method, so they will not be further described. After the multi-modeling module 624 of the arterial pulse analysis system 6 obtaining the feature points of the pulse, the indicator calculation module 625 further performs calculations, based on the feature points obtained, the cardiovascular health can be assessed and the assessment results are displayed via the display unit 63 (e.g., a monitor).

With the arterial pulse analysis method and system provided in this disclosure, non-standard arterial pulse signals with patterns such as monotonically decrease or local oscillations can be processed to widen the applications of arterial pulse analysis technique. In addition, the locations of the feature points in the waveform of the arterial pulse signal can be identified for each heartbeat, and the feature points can be used to assess the cardiovascular health of the user. Moreover, the multi-modeling algorithm is used in conjunction with Maximum Likelihood estimation and Expectation Maximization to reduce the time required for converging a Gaussian function, thereby greatly reducing the processing time of the arterial pulse analysis method, which can be widely applied to arterial pulse measuring devices to enhance the efficiency for retrieving the feature points of the arterial pulse and more precisely assess the cardiovascular health.

It will be apparent to those skilled in the art that various modifications and variations can be made to the disclosed embodiments. It is intended that the specification and examples be considered as exemplary only, with a true scope of the disclosure being indicated by the following claims and their equivalents.

1. An arterial pulse analysis method, comprising:
obtaining a continuous pulse signal through an arterial pulse measuring device;
segmenting the continuous pulse signal into a plurality of single pulses;
performing a data pre-processing step on at least one of the single pulses to obtain non-time series data corresponding to the at least one of the single pulses; and
processing the non-time series data of the at least one of the single pulses with a multi-modeling algorithm to obtain at least one feature point corresponding to the at least one of the single pulses.

2. The arterial pulse analysis method of claim 1, wherein the data pre-processing step includes:
adjusting a baseline of an amplitude of the at least one of the single pulses to a positive value; and
segmenting the at least one of the single pulses unit time by unit time and converting a value of the amplitude of the at least one of the single pulses to form the non-time series data of the at least one of the single pulses.

3. The arterial pulse analysis method of claim 2, wherein the value of the amplitude of the at least one of the single pulses is converted by amplifying or reducing the value.

4. The arterial pulse analysis method of claim 1, wherein the multi-modeling algorithm uses a mixture model of at least one Gaussian model and at least one triangular wave model, a Gaussian mixture model of at least two Gaussian functions, or a plurality of triangular wave models to process the non-time series data of the at least one of the single pulses.

5. The arterial pulse analysis method of claim 4, wherein the multi-modeling algorithm further includes Maximum Likelihood estimation and Expectation Maximization to converge the mixture model, the Gaussian mixture model, or the triangular wave models.

6. The arterial pulse analysis method of claim 4, wherein the feature point corresponds to an intersection of the Gaussian model and the triangular wave model in the mixture model, a characteristic value of the Gaussian model in the mixture model, a characteristic value of the triangular wave model in the mixture model, a characteristic value of the Gaussian functions in the Gaussian mixture model, or a characteristic value of the triangular wave models.

7. The arterial pulse analysis method of claim 1, further comprising, after obtaining the continuous pulse signal, performing a filtering step on the continuous pulse signal, wherein the filtering process includes high-pass filtering, low-pass filtering, or bandpass filtering.

8. The arterial pulse analysis method of claim 1, wherein the continuous pulse signal is segmented into the single pulses based on valleys or peaks of the continuous pulse signal.

9. The arterial pulse analysis method of claim 1, wherein the arterial pulse measuring device is a sphygmomanometer, a sphygmography, an oximeter, or a camera.

10. The arterial pulse analysis method of claim 1, wherein the feature point includes at least one of a pacemaker, a percussion wave peak, a dicrotic notch, and a dicrotic wave peak.

11. An arterial pulse analysis system, comprising:
a signal acquisition unit for generating a continuous pulse signal; and
an operation unit, including:
a pulse segmentation module for processing the continuous pulse signal to segment the continuous pulse signal into a plurality of single pulses;
a pre-processing module for processing at least one of the single pulses to obtain non-time series data corresponding to the at least one of the single pulses; and
a multi-modeling module for processing the non-time series data of the at least one of the single pulses to obtain at least one feature point corresponding to the at least one of the single pulses.

12. The arterial pulse analysis system of claim 11, wherein the operation unit further includes a filter module for receiving and filtering the continuous pulse signal after the signal acquisition unit has generated the continuous pulse signal.

13. The arterial pulse analysis system of claim 11, wherein the pre-processing module adjusts a baseline of an amplitude of the at least one of the single pulses to a positive value, and then segments the at least one of the single pulses unit time by unit time and converts a value of the amplitude of the at least one of the single pulses to obtain the non-time series data corresponding to the at least one of the single pulses.

14. The arterial pulse analysis system of claim 11, wherein operation unit further includes an indicator calculation module for performing cardiovascular health assessment based on the feature point and generating an assessment result.

15. The arterial pulse analysis system of claim 14, further comprising a display unit for displaying the assessment result generated by the indicator calculation module.
